(19) United States
(12) Patent Application Publication
(10) Pub. No.: US 2025/0271366 A1
(43) Pub. Date: Aug. 28, 2025

(54) SYSTEM AND METHOD FOR IMPROVING X-RAY PROJECTION IMAGING LIMIT

(71) Applicant: Gu Xu, Hamilton (CA)

(72) Inventors: Gu Xu, Hamilton (CA); Kelvin J. Xu, Hamilton (CA)

(21) Appl. No.: 18/589,024

(22) Filed: Feb. 27, 2024

(51) Int. Cl. G01N 23/046

(52) U.S. Cl. CPC G01N 23/046; G01N 2223/1016; G01N 2223/313; G01N 2223/401

(57) ABSTRACT

The present disclosure provides an advanced imaging system and method for enhancing the resolution of X-ray images beyond traditional limits. The system utilizes a rectangular aperture X-ray source to project an X-ray beam through one or more pinholes, creating projections of rectangles on a detector screen. The number and overlap of these rectangles depend on the pinholes configuration. A processing unit, employs a two-dimensional difference method to decipher the images. The method isolates the corners of the rectangles, significantly smaller than the original rectangle size, thus enhancing the resolution. While a general sample can be visualized as a collection of pinholes, with more or less penetration capacity, the method is capable of achieving micrometer-scale resolution and potentially nanometer-scale resolution by using shorter X-ray wavelengths.

### FIELD OF THE INVENTION

The present invention relates to X-ray projection systems, and more specifically, to a system and method for improving resolution limit of medical X-ray projection imaging to the micrometer level. The system uses sub-millimeter sized rectangular aperture for X-ray, instead of the conventional circular beam. More specifically, the present invention uses a combination of X-ray optics manipulation and digital imaging deciphering to improve the resolution limit.

### BACKGROUND OF THE INVENTION

The following description includes information that may be useful in understanding the present disclosure. It is not an admission that any of the information provided herein is prior art or relevant to the presently claimed invention, or that any publication specifically or implicitly referenced is prior art.

Projection imaging by x-ray or others has been used extensively in radiography for medical diagnosis and structural examinations, because of its penetration power through the opaque human body and non-transparent packages. However, the image resolution has always been limited by the size of the light source, usually in the sub-millimeter ranges, being too coarse for biological cell identifications, which typically requires a resolution of micrometers. This is simply caused by the dilemma that, the smallest pixel attainable, is correlated to the finite size of the light source. The latter cannot be too small, or the intensity becomes too weak to perform the diagnosis, within a limited time window for a patient to stay still, in the cases of chest x-ray, mammogram, or even computed tomography (CT). Further, there are challenges in determining and setting the size of the light source and intensity of the X-rays emitted by the light source. For example, If the light source is too small, the intensity of the emitted X-ray becomes too weak to provide a clear diagnostic image. Another practical constraint is the time window during which a patient can remain still for X-ray projection imaging. This is especially relevant in diagnostic procedures where even minor movements can affect the quality of the image. Therefore, the intensity of the X-ray needs to be sufficiently high to capture a clear image in a short time.

X-rays have extremely short wavelengths (sub-nanometer) and coherent lengths of less than a micron. Thus, X-rays can capture very fine details, theoretically allowing for high-resolution imaging through mere projection. X-rays can capture very fine details, theoretically allowing for high-resolution imaging through mere projection, without worrying about the diffraction. However, an X-ray emitter, such as a tube anode in an X-ray machine, can be considered a collection of point sources. Each of the point sources emits X-rays that travel through a sample (such as a human body) and reach the detector (such as X-ray film). Since the beams from different point sources are superimposed on each other at the detector, a blurred image is created. Theoretically, the blurriness could be resolved using the point spread function (PSF) of the X-ray source. However, resolving blurriness through PSF is impractical as PSF transform often involves an oscillation function with many zeros, making the mathematical operation (division) challenging or unfeasible.

Alternative solutions such as reducing the size of the X-ray source to minimize blurriness are also implemented by users. However, it reduces the flux (intensity of X-ray beams), which in turn increases the exposure time. This reduces the usability of the projection in patient diagnosis.

To overcome the above challenges associated with use and of conventional X-ray projection imaging using X-ray sources, there is a need for resolving the pixels hidden within one blurred spot sized by the X-ray light source.

### SUMMARY OF THE INVENTION

The present disclosure overcomes one or more shortcomings of the prior art and provides additional advantages discussed throughout the present disclosure. Additional features and advantages are realized through the techniques of the present disclosure. Other embodiments and aspects of the disclosure are described in detail herein and are considered a part of the claimed disclosure.

In one aspect, the present disclosure uses a square or rectangular aperture beam instead of using a conventional circular or irregular cross-sectioned source for the X-ray beam. The square or rectangular aperture through which X-rays are emitted influences the pattern of the X-ray beam and, consequently, the image formed on the detector.

In another aspect, the square or rectangular aperture leads to a different blurry pattern compared to a circular or irregular one. The blurry pattern created by the square or rectangular aperture is simpler to process computationally.

In one exemplary embodiment of the present disclosure, the imaging system includes a rectangular aperture X-ray source configured to project an X-ray beam of size ‘D’ through one or more pinholes, resulting in the projection of one or more rectangles on a detector screen. A digital detector is configured to capture images of the projected rectangles. A processing unit is configured to apply a two-dimensional difference method to decipher the captured images into a plurality of smaller dots of size ‘d’, wherein the deciphering process includes isolating corners of the rectangles, which are significantly smaller than the original rectangle size ‘D’. While a general sample can be visualized as a collection of pinholes, with more or less penetration capacity, the method is capable of achieving micrometer-scale resolution imaging. In one embodiment, two-dimensional difference method can be implemented using the function:
image data matrix=p(1:N, 1:N); dp=p(1:N−d,1:N−d)−p(1:N−d,d+1:N)+p(d+1:N,d+1:N)−p(d+1:N,1:N−d)

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Further aspects and advantages of the present disclosure will be readily understood from the following detailed description with reference to the accompanying drawings, where like reference numerals refer to identical or functionally similar elements throughout the separate views. The figures together with the detailed description below, are incorporated in and form part of the specification, and serve to further illustrate the aspects and explain various principles and advantages, in accordance with the present disclosure wherein:

FIG. 1 illustrates a schematic view of use of a rectangular aperture X-ray source to enhance the image resolution, according to embodiments of the disclosed invention;

FIG. 2 illustrates a schematic illustration of the deciphering process used to resolve one pinhole from the rectangle on the detector screen, in accordance with one embodiment of the present invention;

FIGS. 3a-c illustrate use of a rectangular aperture for an improved resolution, in accordance with one embodiment of the present invention;

FIGS. 4a-4d illustrate process of improving resolution of the letter K using fine detector and deciphering image in accordance with one embodiment of the present invention; and

FIGS. 5a-5d illustrate process of improving resolution of the letters SC using fine detector and deciphering image in accordance with one embodiment of the present invention.

While the invention is susceptible to various modifications and alternative forms, specific embodiments thereof are shown by way of example in the drawings and are herein described in detail. It should be understood, however, that the drawings and detailed description thereto are not intended to limit the invention to the particular form disclosed, but on the contrary, the intention is to cover all modifications, equivalents and alternatives falling within the scope of the present disclosure.

### DETAILED DESCRIPTION

There are a great many possible implementations of the invention, too many to describe herein. Some possible implementations are described below. In the following description, for purposes of explanation, numerous specific details are set forth in order to provide a thorough understanding thereof. It should be clear, however, that the innovation can be practiced without various specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, any particular embodiment need not have all the aspects or advantages described herein. Thus, in various embodiments, any of the features described herein from different embodiments may be combined. It cannot be emphasized too strongly, however, that these are descriptions of implementations of the invention, and not descriptions of the invention, which is not limited to the detailed implementations described in this section but is described in broader terms in the claims.

The foregoing has broadly outlined the features and technical advantages of the present disclosure in order that the detailed description of the disclosure that follows may be better understood. It should be appreciated by those skilled in the art that the conception and specific embodiment disclosed may be readily utilized as a basis for modifying or designing other structures for carrying out the same purposes of the present disclosure.

In the present disclosure, the term “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, “include”, “including”, or any other variations thereof, are intended to cover a non-exclusive inclusion, such that a device that comprises a list of components does not include only those components but may include other components not expressly listed or inherent to such setup or device. In other words, one or more elements in a system or apparatus proceeded by “comprises . . . a” does not, without more constraints, preclude the existence of other elements or additional elements in the system or apparatus.

The novel features which are believed to be characteristic of the disclosure, both as to its system and method of operation, together with further objects and advantages will be better understood from the following description when considered in connection with the accompanying Figures. It is to be expressly understood, however, that each of the Figures is provided for the purpose of illustration and description only and is not intended as a definition of the limits of the present disclosure.

The present disclosure aims to overcome the problems associated with conventional X-ray sources and limited image resolution.

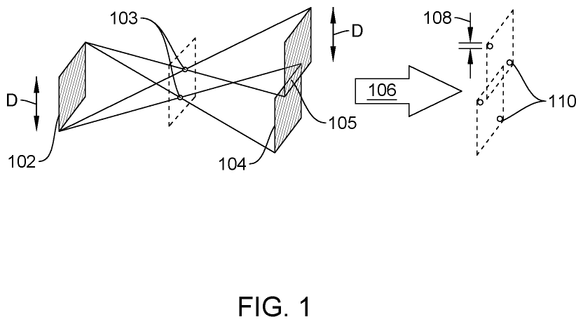

FIG. 1 illustrates a schematic view of use of a rectangular aperture X-ray source to enhance the image resolution, according to embodiments of the disclosed invention. A light source formed by rectangular aperture 102 of size ‘D’ is projected through pinholes 103, resulting in a projection shown through at least one rectangle 104 on a detector screen. The rectangles indicate a blurred image and the number of rectangles 104 can depend on the number of pinholes 103. The rectangles 104 can have a partial overlap 105 depending on the position and orientation of the pinholes 103. The resolution limit of the rectangles 104 is restricted to “D” which is determined by the size of light source 102. The at least one rectangle 104 is then deciphered using a processing unit 106 into a plurality of smaller dots 108 of size ‘d’ using a two-dimensional difference method. The deciphering can involve a process where, only the corners 110 of the at least one rectangle 104 are left, which are much smaller than the original rectangle size ‘D’.

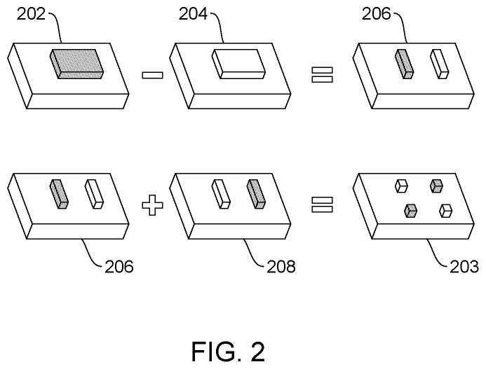

FIG. 2 illustrates a schematic illustration of the deciphering process used to resolve the pinhole from the rectangle on the detector screen, in accordance with one embodiment of the present invention. The deciphering process involves self-subtraction of the horizontally shifted digital data matrix 204 from the original rectangular area 202 of the detector. The step is followed by vertically shifting 206 and adding the inverted 208. As a result, only four corners 203 of the original rectangle 104 remain. Finally, one of the two diagonal pairs will be chopped during the digital display because of their negative values.

The two-dimensional difference method is used for deciphering the projection image from the detector (such as 406,512). The two-dimensional difference method creates a matrix sized 1300*1800, whose value may vary between 0 and 255, depending on the grey-scale of each pixel. The 2-dimensional difference procedure can be applied to the matrix to reduce the rectangles (such as 104) to corner dots, viz. the blurred image from the detector is deciphered to clear sample image.

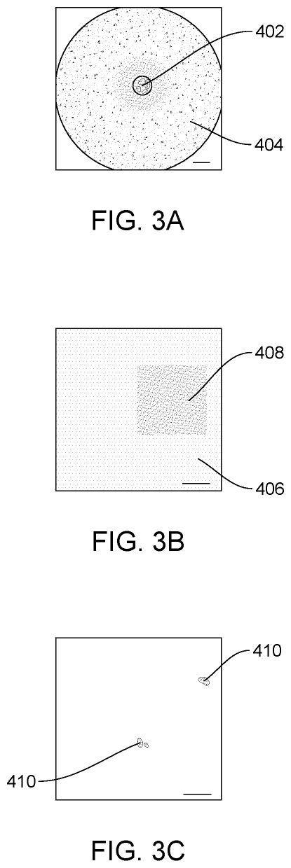

FIGS. 3a-c illustrate use of a rectangular aperture for an improved resolution, in accordance with one embodiment of the present invention. A rectangular aperture of 0.8*0.75 mm is fabricated by lead strips wherein each lead strip is about 0.5 mm thick and installed in front of an X-ray tube, to form a rectangular light source. A pinhole 402 of about 0.1 mm on a lead piece 404 is disposed with the rectangular aperture to ensure a perfect rectangle projection on a detector screen.

FIG. 3b illustrates a finer detector 406 to reduce the size of the projected rectangle 408. By employing the finer detector 406 and the two-dimensional difference method, the rectangle 408 sized ‘D’ on the detector screen 406 is reduced to much smaller dots ‘d’ 410 as illustrated in FIG. 3c. This effectively magnifies the image and improves the resolution limit significantly.

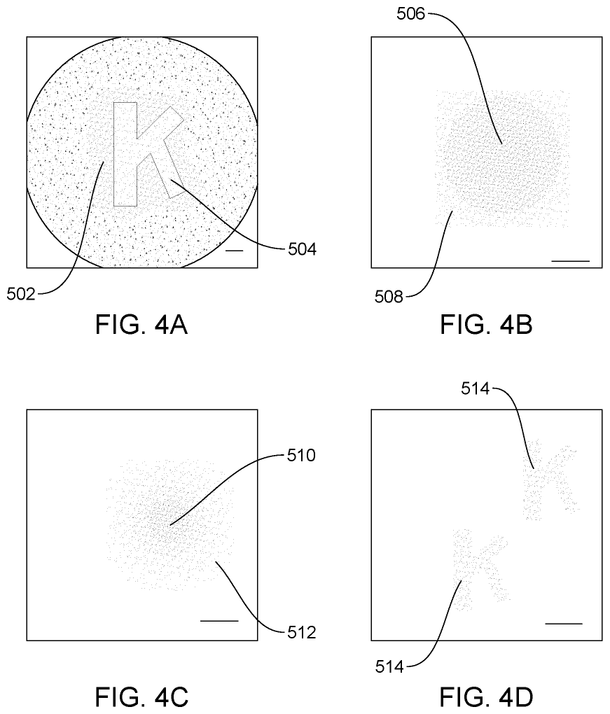

Referring now to FIG. 4a-4d, an optical microscope image 502 of the letter K 504, where the scale bar equals 0.1 mm is shown. FIG. 4b illustrates the projected spots 506 on the detector screen 508 when a coarse detector is employed. The scale bar represents 4 pixels, which equals to 4 mm on the detector, after about 8.8 times enlargement, given by the source-sample-detector distance ratio of 1:8.8.

FIG. 4c illustrates a plurality of rectangles 510 shown on the finer detector 512, and involve hundreds of hidden pixels, where the scale bar represents 200 pixels (20 μm/pixel). Clearly the letter K is still not visible regardless of magnification or detector precision, as the sample size (about 0.5 mm) is smaller than the X-ray source size (0.8 mm). FIG. 4d illustrates the deciphered image 514 after 2-dimensional deference to give a clear letter K. The scale bar represents 200 pixels (20 μm/pixel).

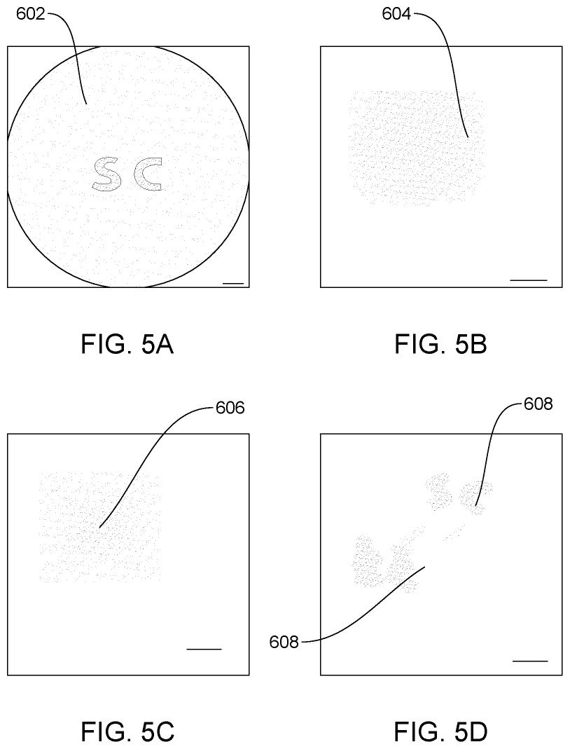

Similarly, FIG. 5a-5d illustrate the optical microscope image 602 of the letters “SC”, sized by 0.7*0.35 mm, where the scale bar equals 0.1 mm. FIG. 5b illustrates the projected spots 604 on the detector screen when a coarse detector was employed. The scale bar represents 4 pixels, which equals to 4 mm on the detector, after about 8.8 times enlargement, given by the source-sample-detector distance ratio of 1:8.8.

FIG. 5c illustrates the projected spots 606 of FIG. 5b but on the fine detector, where the scale bar represents 200 pixels (20 μm/pixel). Finally, FIG. 5d illustrates the deciphered image after 2-dimensional deference, showing a clear image 608 of the letter combo “|SC”, where the scale bar represents 200 pixels (20 μm/pixel).

By using the rectangular aperture and a non-sophisticated image processing technique, the present invention overcomes traditional resolution limits and allows for much finer detail in X-ray images. The present invention not only improves resolution to the micrometer scale but also opens the possibility of reaching nanometer scale resolution with the use of shorter X-ray wavelengths, representing a significant advancement in the field of radiographic imaging.

In one embodiment of the present invention, MSLFP35 intra-oral X-ray image sensor of 1300*1800 pixels is used as the X-ray detector. The pixel of the image sensor has a pitch of 20 μm. The rectangular aperture was fabricated using four lead strips, each lead strip having a thickness of 0.5 mm, with the edge roughness of less than 10 μm. The detector is adapted to align with the light source and sample by the visible light to ensure the projected image is located in the middle of the detector screen.

In different embodiments of the present invention, the pinhole and letter samples are fabricated on 0.5 mm thick lead plates using a laser drill, including punch through letters K, SC, and non-penetrated letter K. The corner size of d, or the ultimate resolution of the strategy outlined here, could not be reduced indefinitely, even with a still finer pixeled detector. It is related to the signal-to-noise ratio (SNR˜D/d). If the noise level is increased, or the edge of the rectangle aperture becomes rough, it accepts only a larger d value in the deciphering process.

While this invention has been described in detail with particular references to embodiments thereof, the embodiments described herein are not intended to be exhaustive or to limit the scope of the invention to the exact forms disclosed. Persons skilled in the art and technology to which this invention pertains will appreciate that alterations and changes in the described structures and methods of assembly and operation can be practiced without meaningfully departing from the principles, and scope of this invention. Additionally, although relative terms such as “upwards,” “downwards,” “turned,” and similar terms have been used herein to describe a spatial relationship of one element to another, it is understood that these terms are intended to encompass different orientations of the various elements and components of the invention in addition to the orientation depicted in the figures. Further, as used herein, when a component is referred to as being “on” or “coupled to” another component, it can be directly on or attached to the other component or intervening components may be present therebetween.

While the invention has been described in connection with what is presently considered to be the most practical and various embodiments, it is to be understood that the invention is not to be limited to the disclosed embodiments, but on the contrary, is intended to cover various modifications and equivalent arrangements included within the spirit and scope of the appended claims.

This written description uses examples to disclose the invention, including the best mode, and also to enable any person skilled in the art to practice the invention, including making and using any devices or systems and performing any incorporated methods. The patentable scope of the invention is defined in the claims and may include other examples that occur to those skilled in the art. Such other examples are intended to be within the scope of the claims if they have structural elements that do not differ from the literal language of the claims, or if they include equivalent structural elements within substantial differences from the literal languages of the claims.

What is claimed is:

1. An imaging system, comprising:
a rectangular aperture X-ray source configured to project an X-ray beam of a first size through pinholes or general samples, resulting in the projection of overlapping rectangles on a detector screen;
a digital detector configured to capture images of the projected rectangles; and
a processing unit configured to decipher the captured images into a plurality of smaller dots to form finer images, wherein the deciphering process includes isolating corners of the rectangles.

2. The imaging system of claim 1, wherein the number of rectangles on the detector screen is dependent on the number of pinholes or general samples used in the projection.

3. The imaging system of claim 1, wherein the processing unit employs a self-subtraction and shifting process to decipher the rectangles into smaller dots.

4. The imaging system of claim 1, wherein the rectangular aperture is fabricated using lead strips or other X-ray absorbing materials.

5. The imaging system of claim 1, wherein the system is configured to enhance the resolution to the micrometer scale and potentially to the nanometer scale by employing shorter X-ray wavelengths.

6. A method for enhancing image resolution of a projected image, comprising:
providing an imaging system, the imaging system includes a rectangular aperture, a digital detector, and a processing unit;
projecting an X-ray or neutron beam through at least one pinhole or general sample using the rectangular aperture, wherein the projecting results in one or more overlapping projected rectangles on a detector screen;
capturing images of the projected rectangles using the digital detector; and
deciphering, by the processing unit, the captured images into a plurality of smaller dots, wherein the deciphering includes isolating corners of the projected rectangles.

7. The method of claim 6, wherein the step of projecting an X-ray beam includes using pinholes or general samples to generate overlapping rectangles on the detector screen.

8. The method of claim 6, further comprising the step of employing a finer detector to reduce the size of the projected rectangles and enhance the resolution of the final image.
